(19) United States
(12) Patent Application Publication
(10) Pub. No.: US 2003/0138508 A1
(43) Pub. Date: Jul. 24, 2003

(54) Method for administering an analgesic

(76) Inventors: Gary D. Novack, San Rafael, CA (US); Stephen A. Schneider, Palo Alto, CA (US)

Correspondence Address: Mika Mayer, Morrison & Foerster LLP, 755 Page Mill Road, Palo Alto, CA, 94304-1018, US

(21) Appl. No.: 10/322,227

(22) Filed: Dec. 17, 2002

Related U.S. Application Data
(60) Provisional application No. 60/342,066, filed on Dec. 18, 2001.
(60) Provisional application No. 60/412,068, filed on Sep. 18, 2002.

(51) Int. Cl. A61K 35/78; A61K 31/445; A61K 31/353; A61K 31/16

(52) U.S. Cl. 424/774; 514/317; 514/454; 514/625

(57) ABSTRACT

A method is provided to parenterally administering an analgesic (i.e., fentanyl) to a patient in the presence of a cannabinoid receptor agonist. This has been found to unexpectedly result in an almost order of magnitude increase in the therapeutic index over that of administering fentanyl alone. The respective amounts of the cannabinoid receptor agonist and fentanyl are selected to achieve the therapeutic index of the analgesic is greater than about 1000. While the method of the present invention contemplates administering the drug by all the medication routes other than orally, the preferred route is via inhalation.

### CROSS-REFERENCE TO RELATED APPLICATIONS

This application claims priority to U.S. provisional application Serial No. 60/342,066 entitled “Method for Administering an Analgesic,” filed Dec. 18, 2001, Gary Novak and Stephen A. Schneider, the entire disclosure of which is hereby incorporated by reference. This application further claims priority to U.S. provisional application Serial No. 60/412,068 entitled “Method for Administering an Analgesic,” filed Sep. 18, 2002, Gary D. Novack and Stephen A. Schneider, the entire disclosure of which is hereby incorporated by reference.

### FIELD OF THE INVENTION

This invention relates to a method for parenterally administering to a patient an analgesic in the presence of a cannabinoid receptor agonist.

### BACKGROUND OF THE INVENTION

It is well known that THC and other extracts of cannabinoid affect both peripheral and central nervous system activity. Behavioral effects of such compounds are characterized at low doses as a mixture of depressant and stimulatory effects and at higher doses as predominantly CNS depressants (Dewey, 1986). The depressant effects of cannabinoids produce hyperreflexia. Cannabinoids generally cause a reduction in spontaneous locomotor activity and a decrease in response rates. Cannabinoids also impair learning and memory in rodents and non-human primates. Other effects that have been shown in the mouse include hypothermia (Compton et al., 1993), immobility (catalepsy) and antinociception, which comprise the “tetrad” of tests for cannabinoid activity (Martin, 1985). The mechanisms which underly the other effects of the cannabinoids as tested in the “tetrad” have been shown to be pertussis toxin-senstitive (Lichtman et al., 1996) and thus, are likely mediated via G-protein activation.

Recent articles summarize the extensive evaluation of the analgesic and antinociceptive effects of the cannabinoids (Martin and Lichtman, 1998) and the neural substrates mediating such responses (Walker et al., 1999). Early experiments to evaluate the analgesic effects of the cannabinoids dealt mainly with an examination of the effects of THC, the principle active ingredient in cannabis. Studies in human subjects indicate that at oral doses of 10 and 20 mg/kg THC was no more effective than codeine as an analgesic, while producing a significant degree of dysphoria side effects (Noyes et al., 1975). When tested following intravenous administration to human dental patients, THC produced antinociception that was accompanied by dysphoria and anxiety (Raft et al., 1977). Thus in these studies it was evident that THC analgesia could only be elicited at doses producing other behavioral side effects. In addition, THC was no more potent than more commonly used opioid analgesics.

Cannabinoids are active as analgesic drugs when administered to laboratory animals by several routes of administration (Yaksh, 1981; Gilbert, 1981; Lichtman and Martin, 1991 a and b; Welch and Stevens, 1992, Welch et al., 1995a). Early studies by Sofia et al. (1973) and Moss and Johnson (1980) established that THC administered orally (p.o.) is effective in the rat paw pressure test. Similarly, it has been shown that the synthetic cannabinoid, WINN 55,212-2, alleviates the pain associated with sciatic nerve constriction in rats (Herzberg et al. 1997), capsaicin-induced hyperalgesia in rats (Li et al., 1999) and in rhesus monkeys (Ko and Woods, 1999). Cannabinoid-induced antinociception appears to be produced by the inhibition of wide dynamic range neurons in the spinal cord dorsal horn (Hohmann et al., 1999). The endogenous cannabinoid system appears to be an active component of chronic pain in that the CB 1 antagonist, SR141716A, has been shown to produce hyperalgesia in rats (Strangman et al., 1998; Martin et al., 1999) and mice (Richardson et al., 1997 and 1998).

Recently the interaction of cannabinoids with certain opioids has been extensively reviewed (Cichewicz et al., “Enhancement of μ Opioid Antinociception by Oral Δ⁹-Tetrahydrocannabinol: Dose-Response Analysis and Receptor Identification,” The Journal of Pharmacology and Experimental Therapeutics, Vol. 289, pp. 859-867, 1999). The latter article reported that μ opioids were found to be enhanced by an inactive dose of Δ⁹-THC when taken p.o. One of the opioids tested was fentanyl. Although fentanyl was enhanced by Δ⁹-THC based on an ED₅₀, the median effective dose that produces the desired effect in 50% of the animals tested, the article stated that doses higher than 1 mg/kg could not be tested because of its toxicity in animals.

Administering fentanyl p.o. tends to be less effective than parenterally because the drug must first be absorbed from the gastrointestinal tract and then delivered to the liver. This is the case because the liver extensively metabolizes fentanyl. Thus, administering fentanyl parenterally causes the drug to travel directly from its site of entry, a vein in the case of intravenously (i.v.), to the brain, its primary site of action, before it passes through the liver. The administration of fentanyl to patients is currently provided in several dosage forms: intravenous, transdermal and transmucosal. The latter consists of a matrix of fentanyl citrate on a stick (Actiq® oral transmucosal fentanyl citrate). The product literature provided for Actiq indicate that 25% of the dose is absorbed from the buccal mucosa while the remaining 75% is swallowed with the saliva and is then slowly absorbed from the gastrointestinal tract. About ⅓ of this amount (25% of the total dose) escapes hepatic and intestinal first-pass elimination and becomes systemically available. It has long been known that fentanyl, no matter how it is administered, must be done with great care to avoid toxicity. Therefore, one skilled in the art would be directed away from parenterally administering fentanyl in the presence of THC, or other cannabinoid receptor agonist, because of the problem of toxicity as discussed in the foregoing Cichewicz et al. article.

The present invention overcomes the toxicity problem by greatly lowering the amount of fentanyl required to achieve an effective analgesic dose and dramatically increasing the amount of fentanyl that can be administered without toxicity. In other words, the therapeutic index of fentanyl is profoundly expanded, an unexpected and heretofore unexplored phenomenon.

### SUMMARY OF THE INVENTION

Embodiments of the present invention are directed to a method of parenterally administering fentanyl in the presence of a cannabinoid receptor agonist (e.g., THC or other cannabinoid extracts) to a patient, which unexpectedly results in an almost order of magnitude increase in the therapeutic index over that of administering fentanyl alone. The respective amounts of the cannabinoid receptor agonist and fentanyl are determined so that the therapeutic index of the analgesic is greater than about 1000.

The therapeutic index (TI) is the ratio of LD50/ED50, where LD50 is the median lethal dose that will kill 50% of the animals receiving that dose and ED50 is defined above. The higher the TI the more unlikely it will be for the administration of the analgesic dose of a drug to produce toxicity in terms of lethality.

A cannabinoid receptor agonist is a composition or compound possessing a Kᵢ (nM) for either the CB₁ or CB₂ receptors that is less than 1000. Preferably, the agonist will possess a Kᵢ (nM) for the CB₁ receptor that is less than 500. More preferably, the agonist will possess a Kᵢ (nM) for the CB₁ receptor that is less than 100.

The method of the present invention comprises parenterally administering fentanyl and a cannabinoid receptor agonist to a patient, wherein the amounts of administered fentanyl and cannabinoid receptor agonist are selected such that the therapeutic index of fentanyl in the presence of the cannabinoid receptor agonist is greater than about 1000. The cannabinoid receptor agonist can be in a vehicle.

Typically, the fentanyl is administered by one of the following routes: intravenously, subcutaneously, intrathecally, transdermally, and through inhalation. Preferably, it is administered intravenously, transdermally or through inhalation.

Typically, the cannabinoid receptor agonist is selected from a group consisting of a cannabinoid extract, 11-hydroxy-Δ⁸-THC-dimethylheptyl, CP 55940, CP 55244, CP 50556, desacetyl-L-nantradol, WIN 55,212-2, and anandamide. Preferably, the cannabinoid receptor agonist is a cannabinoid extract.

Typically, the cannabinoid extract is selected from a group consisting of cannabis, tetrahydrocannabinol, and cannabis/tetrahydrocannabinol mixtures. Preferably, the cannabinoid extract is tetrahydrocannabinol.

Typically, where fentanyl is administered through inhalation, it is administered as an aerosol. Preferably, the aerosol is at least 50 percent by weight of fentanyl. More preferably, the aerosol is at least 75, 90, 95, or 97.5 percent by weight of fentanyl.

Typically, where the cannabinoid receptor agonist is administered through inhalation, it is administered as an aerosol. Preferably, the aerosol is at least 50 percent by weight of a cannabinoid receptor agonist. More preferably, the aerosol is at least 75, 90, 95, or 97.5 percent by weight of a cannabinoid receptor agonist.

Typically, where the fentanyl is administered as an aerosol, the aerosol is formed by heating a composition comprising fentanyl. Preferably, the composition comprising fentanyl is at least 95 percent by weight of fentanyl.

Typically, where the cannabinoid receptor agonist is administered as an aerosol, the aerosol is formed by heating a composition comprising the cannabinoid receptor agonist. Preferably, the composition comprising the cannabinoid receptor agonist is at least 95 percent by weight of cannabinoid receptor agonist.

In one embodiment of the present method, fentanyl and the cannabinoid extract are respectively heated to vaporize at least a portion of each of the compounds, the resulting vapors are mixed with a gas (e.g., air), and the resulting aerosol is administered to the patient.

### BRIEF DESCRIPTION OF THE DRAWINGS

Further features and advantages will become apparent from the following description of various embodiments of the invention, as illustrated in the accompanying drawings in which:

FIG. 1 is a dose response curve for administering fentanyl alone;

FIG. 2 is a dose response curve for administering Δ⁹-THC alone; and

FIG. 3 is a dose response curve for administering a combination of fentanyl and Δ⁹-THC.

### DETAILED DESCRIPTION

The method of the present invention results in a TI over 1000 by selecting an amount of fentanyl in the range of about 0.001 to about 0.1 mg per kg (typically, 0.005 to about 0.1 mg per kg) of body weight of the patient and an amount of the cannabinoid receptor agonist in an amount in the range of about 0.01 to about 1.0 mg per kg (typically, 0.1 to about 1.0 mg per kg) of the body weight.

While the method of the present invention contemplates administering the combination of fentanyl and cannabinoid receptor agonists by all the medication routes other than orally, there is a significant advantage of using inhalation as the route because it provides a means for rapid absorption of drugs such as fentanyl into the blood system for delivery directly to the brain, without the use of needles or excipients or other vehicles and without being exposed to a first pass metabolism in the gastrointestinal tract or liver.

In a preferred embodiment of the present invention, fentanyl and the cannabinoid receptor agonist are volatilized into vapors avoiding medicinally-significant degradation and thus maintaining acceptable compound purity by heating the compounds to a volatilizing temperature for a limited time.

Fentanyl decomposes rapidly at 300° C. before reaching its boiling point and can be vaporized in quantities up to 2 mg at temperatures around 190° C. Vaporization can therefore be accomplished at practical rates, i.e., in the range of about 0.5 to about 2 mg/second, and at temperatures much below the compound's boiling points. The ability to vaporize at these reduced temperatures provides a means to lower the rates of degradation reactions in many compounds including fentanyl and cannabinoid receptor agonists such as THC. Specifically, 100% of a fentanyl sample decomposed when heated to 200° C. for 30 seconds, but decreased to 15-30% decomposition when fentanyl was heated to 280° C. for 10 milliseconds.

When fentanyl was vaporized using a laboratory device, which minimized the vaporization temperature and limited the exposure time to that temperature, no medicinally significant decomposition (<0.1%) was observed. The laboratory device and the method of administering fentanyl are disclosed and claimed in U.S. application Ser. No. 10/057,197, filed Oct. 26,2001 (Docket Number 6577-60341), the description of which is incorporated herein by reference. The laboratory device was successfully used to deliver experimental doses of an aerosol comprising fine particles of fentanyl in amounts ranging between 20 μg and 500 μg suspended in about 800 cc of air to 10 kg dogs under test. A comparison was made between administering fentanyl via i.v. and using this laboratory device on the same type of dogs. One set of three dogs received fentanyl at a 100 μg intravenous bolus dose. The same dogs received fentanyl in an ultra fine (UF) aerosol for inhalation (100 μg aerosolized and administered as two successive activations of this laboratory device, containing approximately 50 μg of fentanyl base). The results of the comparison determined that the time course of inhaled fentanyl was nearly identical to that of i.v. fentanyl. Thus, fentanyl UF for inhalation had an exposure profile that was found to be similar to that of an i.v. injection.

### EXAMPLES

The following examples further illustrate the method of the present invention. These examples are for illustrative purposes and are not meant to limit the scope of the claims in any way.

### Example 1

Male ICR mice from Harlan Laboratories, Indianapolis, Ind. weighing 25 to 30 grams were housed in a group of 6 per cage in an animal care facility maintained at 22±2° C. on a 12-hour light/dark cycle. Food and water were available on demand throughout the experiments. This protocol is fully authorized under the University Animal Care and Use Committee Protocol #0109-2986 (renewal date Nov. 30, 2001).

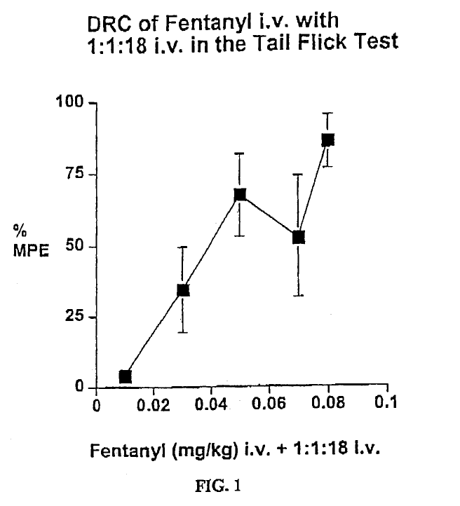

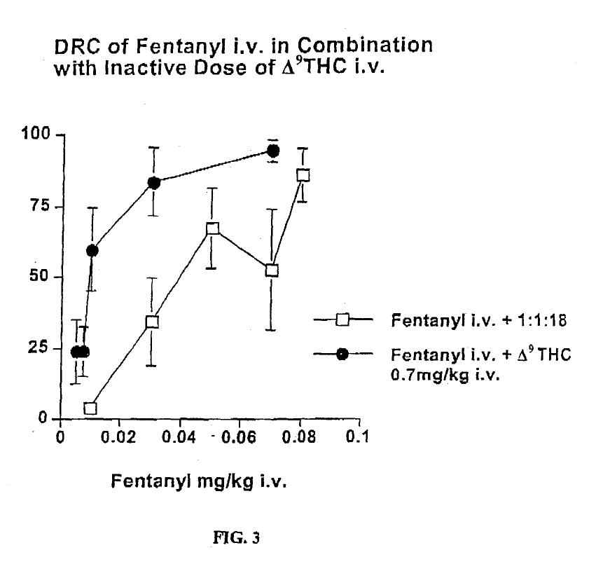

The mice were brought to the test room and allowed to acclimate for 24 hours to recover from transportation and handling. For the generation of dose response curves (DRC) in FIGS. 1-3 for fentanyl alone, Δ⁹-THC alone, and a combination of fentanyl with Δ⁹-THC. All of the drugs were administered intravenously (i.v.) during this example. Fentanyl was in the form of fentanyl citrate obtained from Sigma Chemical Co. (St. Louis, Mo.) and was dissolved in saline. Δ⁹-THC was obtained from the National Institute on Drug Abuse (Rockville, Md.) and was prepared in a vehicle of emulphor, ethanol, and saline at a 1:1:18 ratio. The drugs were i.v. injected at 10 minutes prior to testing in a tail-flick test for antinociception. Injections were into the lateral tail veins of each mouse, one injection per vein. The injection volume was 0.1-cc/10 gm of body weight.

The tail-flick test, also known as the spinal reflex test, was designed by D'Amour and Smith, “A Method for Determining Loss of Pain Sensation,” J. Pharmacol. Exp. Ther., Vol. 7, pp 274-279, 1941. In the test, each mouse was exposed to radiant heat on its tail. When the heat became nociceptive, the mouse freely escaped from the pain by flicking its tail. The baseline values in seconds prior to testing were 2 and 4 seconds. A cut-off of 10 seconds was employed to prevent burns. The % MPE (percent maximum possible effect) for each mouse was calculated as described above using the formula developed by Harris and Pierson, “Some Narcotic Antagonists in the Benzomorphan Series,” J. Pharmacol. Exp. Ther., Vol. 7, pp 141-148, 1964:

% MPE=[test (sec)−control (sec)/10−control]×100.

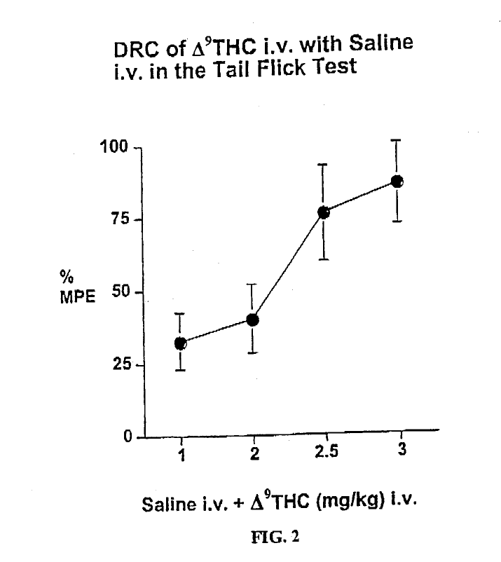

The % MPE for each mouse was entered into the Tallarida and Murray ED₅₀ software program (1986). The ED₅₀ was calculated along with 95% confidence intervals [CL's]. At least 6 mice were used for each dose and treatment. ED₅₀'s are determined to be significantly different from each other if the 95% confidence limits do not overlap. The inactive dose of THC was 0.7 mg/kg as determined from the dose-response curve (DRC) of THC shown in FIG. 2. This inactive amount was used in combination with fentanyl in experiments of this example. The ED₅₀ values and 95% CL's were determined using unweighted least-squares linear regression for the log dose-response curves as described by Tallarida and Murray, Procedures 6, 8, 9, 11, in Manual of Pharmacologic Calculations With Computer Programs, Springer-Verlag, New York, 1987.

The LD₅₀ was performed using the following injection protocol. The number of deaths per group of 6 mice was calculated for each of the different types of groups listed below. The % lethality was calculated as [# of dead/6]×100. LD₅₀ was determined as per Tallarida and Murray LD₅₀ software program.

The groups tested:

1. Dose-response fentanyl+[1:1:18 vehicle described above]

2. Dose-response THC+[saline vehicle}

3. Dose-response fentanyl+THC [0.7 mg/kg]

4. Control group of 6 mice: vehicle [saline]+vehicle [1:1:18]

The therapeutic index (TI) was calculated based on the LD₅₀/ED₅₀ per standard calculations from the Tallarida and Murray program.

The results of this example are set forth in Table 1 below:

TABLE 1
ED₅₀ and LD₅₀ Values and TI for Fentanyl, THC, and Fentanyl/THC
Combination

| Drug | ED₅₀[95% CL's] | LD₅₀[95% CL's] | TI |
|---|---|---|---|
| Fentanyl | 0.04 [0.03-0.06] mg/kg | 23.6 [20-28] mg/kg | 590 |
| THC | 1.6 [1.2-2.2] mg/kg | 75.4 [66.5-85.5] mg/kg | 47 |
| Fentanyl/ | 0.01 [0.008-0.01] mg/kg* | 18 [13.8-23.6] mg/kg | 1800 |
| THC | | | |

*Significantly different from fentanyl alone (no overlap of 95% CL's).

The conclusions that are drawn from the above results are as follows:

1. THC coadministered with fentanyl at its inactive dose of 0.7 mg/kg unexpectedly produced a significant 4-fold shift in the dose-effect curve of fentanyl.

2. THC administered at the inactive dose level unexpectedly increased the TI for fentanyl from 590 to 1800 due to the decrease in ED50 for fentanyl. The LD50 for the fentanyl/THC combination does not differ from fentanyl alone (95% CL's overlap). Surprisingly, THC does not significantly enhance the LD50 of fentanyl.

3. THC has an unexpected order of magnitude lower TI than fentanyl.

4. The combination of fentanyl with a low inactive dose of THC appears to increase the potency and decreases the toxicity of fentanyl.

### Example 2

About 1 mg of Δ⁹-THC was coated onto the stainless steel surface of a flashbar apparatus. (The flashbar is a cylinder 3.5 cm long and 1.3 cm in diameter consisting of a hollow tube of 0.005″ thick stainless steel.) Brass electrodes were connected to either end of the steel cylinder. The coated flashbar was secured in an electrical mount, which connected to two 1.0 Farad capacitors in parallel. An airway was provided by a 2 cm diameter glass sleeve placed around the flashbar. 15 L/min of room air were pulled by a house vacuum through the vaporization chamber and a filter housing, which contained a two-micron Teflon filter. A power supply charged the capacitors to 20.5 volts, at which point the circuit was closed with a switch and the stainless steel flashbar was resistively heated to about 400° C. within about 200 milliseconds. The Δ9-THC aerosolized and flowed through the airway and into the filter. The Teflon filter was extracted with organic solvent, and the sample was run through an HPLC for purity analysis. Purity analysis indicated that the aerosol was approximately 98% Δ⁹-THC (˜87.5% recovery), with cannabinol being the primary impurity.

To obtain higher purity aerosols, one can coat a lesser amount of drug, yielding a thinner film to heat. A linear decrease in film thickness is associated with a linear decrease in impurities.

What is claimed is:

1. A method of treating pain, wherein the method comprises administering fentanyl and a cannabinoid receptor agonist to a patient, and wherein the fentanyl is administered parenterally, and wherein the amounts of administered fentanyl and cannabinoid receptor agonist are selected such that the therapeutic index of fentanyl in the presence of the cannabinoid receptor agonist is greater than about 1000.

2. The method according to claim 1, wherein the fentanyl is administered intravenously.

3. The method according to claim 1, wherein the fentanyl is administered subcutaneously.

4. The method according to claim 1, wherein the fentanyl is administered intrathecally.

5. The method according to claim 1, wherein the fentanyl is administered transmucosally.

6. The method according to claim 1, wherein the fentanyl is administered transdermally.

7. The method according to claim 1, wherein the fentanyl is administered through inhalation.

8. The method according to claim 1, wherein the cannabinoid receptor agonist is selected from a group consisting of a cannabinoid extract, 11-hydroxy-Δ⁸-THC-dimethylheptyl, CP 55940, CP 55244, CP 50556, desacetyl-L-nantradol, WIN 55,212-2, and anandamide.

9. The method according to claim 7, wherein the fentanyl is administered as an aerosol, and wherein the aerosol is at least 50 percent by weight of fentanyl.

10. The method according to claim 9, wherein the aerosol is at least 75 percent by weight of fentanyl.

11. The method according to claim 10, wherein the aerosol is at least 90 percent by weight of fentanyl.

12. The method according to claim 11, wherein the aerosol is at least 95 percent by weight of fentanyl.

13. The method according to claim 12, wherein the aerosol is at least 97.5 percent by weight of fentanyl.

14. The method according to claim 1, wherein the cannabinoid receptor agonist is administered through inhalation.

15. The method according to claim 14, wherein the cannabinoid receptor agonist is administered as an aerosol, and wherein the aerosol is at least 50 percent by weight of the cannabinoid receptor agonist.

16. The method according to claim 15, wherein the cannabinoid receptor agonist is administered as an aerosol, and wherein the aerosol is at least 75 percent by weight of the cannabinoid receptor agonist.

17. The method according to claim 16, wherein the cannabinoid receptor agonist is administered as an aerosol, and wherein the aerosol is at least 75 percent by weight of the cannabinoid receptor agonist.

18. The method according to claim 17, wherein the cannabinoid receptor agonist is administered as an aerosol, and wherein the aerosol is at least 90 percent by weight of the cannabinoid receptor agonist.

19. The method according to claim 18, wherein the cannabinoid receptor agonist is administered as an aerosol, and wherein the aerosol is at least 95 percent by weight of the cannabinoid receptor agonist.

20. The method according to claim 19, wherein the cannabinoid receptor agonist is administered as an aerosol, and wherein the aerosol is at least 97.5 percent by weight of the cannabinoid receptor agonist.

21. The method according to claim 8, wherein the cannabinoid extract is selected from the group consisting of cannabis, tetrahydrocannabinol, and cannabis/tetrahydrocannabinol mixtures.

22. The method according to claim 6, wherein the cannabinoid receptor agonist is selected from a group consisting of a cannabinoid extract, 11-hydroxy-Δ⁸-THC-dimethylheptyl, CP 55940, CP 55244, CP 50556, desacetyl-L-nantradol, WIN 55,212-2, and anandamide.

23. The method according to claim 22, wherein the cannabinoid receptor agonist is tetrahydrocannabinol.

24. The method according to claim 11, wherein the aerosol is formed by heating a composition comprising fentanyl.

25. The method according to claim 24, wherein the composition comprising fentanyl is at least 95 percent by weight of fentanyl.

26. The method according to claim 18, wherein the cannabinoid receptor agonist is tetrahydrocannabinol, and wherein the aerosol is formed by heating a composition comprising tetrahydrocannabinol.

27. The method according to claim 26, wherein the composition comprising tetrahydrocannabinol is at least 95 percent by weight of tetrahydrocannabinol.
